(19) United States
(12) Patent Application Publication
(10) Pub. No.: US 2009/0105194 A1
(43) Pub. Date: Apr. 23, 2009

(54) PROCESS FOR THE PRODUCTION OF A LOW MOLECULAR WEIGHT HEPARIN

(75) Inventors: Ragnar Flengsrud, As (NO); Ole Rasmus Odegaard, Oslo (NO)

Correspondence Address: SUGHRUE MION, PLLC, 2100 PENNSYLVANIA AVENUE, N.W., SUITE 800, WASHINGTON, DC, 20037, US

(73) Assignee: UNIVERSITETET FOR MILJO-OG BIOVITENSKAP, As (NO)

(21) Appl. No.: 11/914,086

(22) PCT Filed: May 9, 2006
(86) PCT No.: PCT/GB06/01690

§ 371 Date: Sep. 18, 2008

(30) Foreign Application Priority Data
May 9, 2005 (GB) 0509433.9

(51) Int. Cl. A61K 31/727; C08B 37/10; A61P 7/02

(52) U.S. Cl. 514/56; 536/55.3

(57) ABSTRACT

The invention provides a process for the production of a very low molecular weight heparin (VLMWH) composition having a VLMWH content, relative to total heparin content, of at least 10% wt, said process comprising chromatographically or chemically or by filtration reducing the relative proportion of heparin having a molecular weight above 8000 Da in a heparin composition extracted from a non-mammalian, vascularised marine animal.

The present invention relates to a process for the production of a very low molecular weight heparin composition.

Heparin is the name given to a class of sulphated glucosaminoglycans having anti-coagulant properties. Heparin is widely used medically both as a coating agent for invasive medical equipment, e.g. catheters and implants, and as therapeutic and prophylactic agents. Moreover heparin has been used in connection with extracorporeal circulational hemodialysis, as an adjunct to chemotherapeutic and anti-inflammatory drugs, as a modulatory agent for growth factors, and in the treatment of haemodynamic disorders, pre-eclampsia, inflammatory bowel disease, cancer, venous thromboembolic disease, unstable coronary ischemic disease, and acute cerebravascular ischemia.

Currently, mammalian tissue, especially from pigs and sheep, is the normal source for commercially available heparin. While previously the most common source was bovine lungs, today the most common source is pigs' intestines.

Heparin has a polymeric structure and thus heparin compositions generally contain heparins having a range of molecular weights typically from 5 kDa to 40 kDA (see for example Mulloy et al., Thromb. Haemost. 84:1052-1056 (2000)). Heparin with this wide range of molecular weights is usually referred to as unfractionated heparin (UFH). As currently used commercially UFH typically has molecular weights in the range 5.0 to 40 kDa. In recent years there has been significant interest in and use of low molecular weight heparin (LMWH), i.e. a material containing heparin, but of low molecular weight, typically less than 8 kDa.

LMWH can be produced from native unfractionated heparin by a variety of processes, e.g. by fractionation or depolymerisation by chemical or enzymatic cleavage, e.g. by nitrous acid depolymerisation or by heparinase digestion. The LMWH currently available is produced from porcine heparin. LMWH generally has a potency of at least 70 units/mg of anti-factor Xa activity and a ratio of anti-factor Xa activity to anti-factor IIa activity of at least 1.5 (see European Pharmacopoeia Commission. Pharmeuropa 1991:3:161-165).

Relative to standard unfractionated heparin (UFH), LMWH has several advantages: it is better absorbed and can be administered subcutaneously; it remains in the blood stream longer; it has a more predictable clinical response; and it may cause fewer of the unwanted side effects that have been associated with UFH, such as excessive bleeding, low platelet count, osteoporosis, and irritation of the injection site. These benefits of LMWH have led to a steady increase in physician preference for LMWH over UFH despite its considerably higher price.

Nonetheless there is a growing concern about the use of UFH or LMWH from mammalian sources in view of the perceived potential for cross-species viral and prion infection. This has led to increased interest in synthetic production of very low molecular weight heparin (VLMWH). Thus biologically active heparin may be made synthetically with a minimal pentameric structure having a molecular weight of about 1.7 kDa.

As currently available, synthetic VLMWH is available from Sanofi-Synthelabo as Arixtra™ or from Alchemia as Synthetic Heparin.

The use of such depolymerisation or synthetic procedures however complicates the production of LMWH and synthetic heparins and makes the end product relatively expensive and hence less available for use by health authorities lacking extensive funding. There is thus a need for a simpler and cheaper route to an effective LMWH or VLMWH.

We have found that heparin extracted from marine animals, in particular fish, naturally has a high content of LMWH and surprisingly also of very low molecular weight heparin (VLMWH), i.e. heparin having a molecular weight less than 3 kDa.

The extraction of marine heparin is described in WO 02/076475, the contents of which are hereby incorporated by reference.

Thus, for example, the LMWH and VLMWH contents of unfractionated heparin from pigs, cattle and salmon gills and waste were found to be as follows:

TABLE 1
LMWH and VLMWH ** contents of UFH

| Source | % wt MW < 8 kDa | % wt MW < 3 kDa |
|---|---|---|
| Pig * | 8.9 | 1.8 |
| Pig intestine *** | 9.6 | 0.4 |
| Cattle * | 2.9 | 0 |
| Salmon gill | 14 ∀ 2 | 6.4 ∀ 0.4 |
| Salmon waste | 12.7 | 8.5 |

* from Sigma
** High antithrombin affinity VLMWH content as determined using the Stachrom Heparin Kit from Diagnostica Stago, Asnieres, France.
*** from LEO Pharma AS

As indicated above, the VLMWH contents for marine heparin tabulated above are contents of VLMWH having high affinity for purified bovine antithrombin. Low affinity VLMWH may also be present and may contribute towards the antithrombotic effect of the products.

VLMWH has benefits over LMWH in the same way as LMWH has advantages over UFH.

Thus in particular it is expected that VLMWH will show prolonged blood half-life, reduced side effects (e.g. thrombocytopenia), and enhanced activity.

In particular we have found that, with marine LWMH, the anti-factor Xa activity of the heparin fraction of molecular weight 1 to 3 kDa is at least 20% higher than that for the 3 to 8 kDa fraction. Moreover the anti-factor Xa activity for individual molecular weight fractions in the range 1 to 3 kDa may be as high as 90 U/mg.

We therefore propose the use of marine heparin as a source material for the production of VLMWH. The marine heparin can be extracted from fish or shellfish waste. Moreover, since the VLMWH content is so high, there is no need for depolymerisation as chromatographic and filtration techniques can be used economically (which is not the case for mammalian UFH). Depolymerisation can however be used if desired.

Thus viewed from one aspect the invention provides a process for the production of a VLMWH composition having a VLMWH content, relative to total heparin content, of at least 10% wt, preferably at least 15% wt, more preferably at least 20% wt, especially at least 25% wt, more especially at least 30% wt (e.g. up to 100% wt, more typically up to 80% wt, for example up to 30% wt), said process comprising chromatographically, enzymatically, chemically or by filtration reducing the relative proportion of heparin having a molecular weight above 8000 Da (particularly that having a molecular weight above 3000 Da) in a heparin composition extracted from a non-mammalian, vascularised marine animal, preferably a fish or shellfish, more preferably from the waste from such an animal after removal of muscle tissue, e.g. for use as a human foodstuff.

The VLMWH content in the compositions produced may be assessed chromatographically, spectroscopically, or using test kits such as the Stachrom Heparin Kit mentioned above.

By non-mammalian marine animal is included fresh-water as well as salt-water fish and shellfish.

Fish used as food sources for mammals or as raw materials for fish meal, fish food, and fish oil are preferred. Particularly preferably farmed fish are used. Examples of suitable fish include: carp, barbell and other cyprinids; cod, hake, haddock; flounder; halibut; sole; herring; sardine; anchovy; jack; mullet; saury; mackerel; snoek; cutlass fish; red fish; bass; eels (e.g. river eels, conger, etc.); paddle fish; tilapia and other cichlids; tuna; bonito; bill fishes; diadromous fish; etc. Particular examples of suitable fish include: flounder, halibut, sole, cod, hake, haddock, bass, jack, mullet, saury, herring, sardine, anchovy, tuna, bonito, bill fish, mackerel, snoek, shark, ray, capelin, sprat, brisling, bream, ling, wolf fish, salmon, trout, coho and chinock. Especially preferably the fish used is trout, salmon, cod or herring, more especially salmon.

The fish waste used as the source for heparin extraction, a step which is an optional precursor step in the process of the invention, will typically be selected from heads, skin, gills, and internal organs. The use of gills alone, of heads and of internal organs is especially preferred. Methods of processing fish waste are known from the literature, e.g. WO2004/049818.

As mentioned above, chemical (or enzymatic) depolymerisation, e.g. using an acid (such as nitrous acid), an alkali, isoamyl nitrite, an oxidant (e.g. hydrogen peroxide or Cu (I)), or a heparinase, may be carried out in the process of the invention. In this regard conventional depolymerisation techniques may be used (see for example Linhardt et al. Seminars in Thrombosis and Hemostasis 25 (suppl 3): 5-16 (1999) and references therein the contents of which are hereby incorporated by reference). Preferably, however, the relative increase in VLMWH content is achieved by filtration (e.g. membrane filtration) or chromatographically, especially preferably using size exclusion chromatography, ion exchange chromatography, or sample displacement chromatography.

Membrane filtration is a well established technique and membranes having particular molecular weight cut-offs are commercially available, e.g. from Pall and Millipore.

Size exclusion chromatography (SEC) is also a well established chemical technique and appropriate separation materials are widely available, e.g. as Sephadex™ or Sephacryl™ from Amersham Biosciences, or Bio-Gel P10, Bio-Gel P30 or Bio-Gel P60 from Bio-Rad. The use of G-75 Sephadex™, Sephacryl™ S-200 HR and Sephacryl™ S-300 HR are especially preferred. It is possible to carry out the SEC step at least twice if desired.

Sample displacement chromatography is described in U.S. Pat. No. 6,245,238 and U.S. Pat. No. 6,576,134, the contents of which are incorporated herein by reference.

In a preferred embodiment of the invention the marine heparin is concentrated and desalted before subjection to the chromatographic step to increase relative VLMWH content. This is especially important when SEC is used. Thus for example the heparin may be separated from other components by loading the heparin-containing material onto an ion exchange column (e.g. a Dowex column) and subsequently releasing it using aqueous saline (e.g. 4M NaCl). The eluate may then be desalted, e.g. using a Millipore/Amicon stirred cell with a Nanomax-50 filter, and then freeze-dried. This removes the salt and minimizes the volume of the redissolved sample to be applied to the SEC column, e.g. a G-75 Sephadex column.

Especially preferably the marine heparin is subjected to membrane filtration to remove low molecular weight components, e.g. with a molecular weight before that of the antithrombin binding pentamer (MW 1728 Da), typically using a membrane with a 1 kDa cut-off (e.g. Omega-1k Ultrasette from Filtron/Pall). Also especially preferably the marine heparin is subjected to membrane filtration to remove high molecular weight components, for example with a molecular weight cut-off of 3000 Da (e.g. using Omega Centramate Suspended Screen OS005C11P1 from Filtron/Pall).

Using ion exchange chromatography, the LMWH and VLMWH content of the product may particularly conveniently be enhanced by applying the sample to the ion exchanger in excess of the exchanger's capacity. Since the low molecular weight heparins are generally the most strongly binding components, their content in the subsequent eluate is correspondingly increased.

The concentrated and desalted heparin may if desired be dried before further handling, e.g. by freeze-drying.

The VLMWH composition produced according to the process of the invention may be dried or may be formulated for use, e.g. with a diluent, carrier or an active drug substance, and it may be applied, preferably after formulation with a liquid carrier, as a coating to the surface of a medical instrument, e.g. a catheter or implant. Such compositions and coated instruments form further aspects of the present invention, as does the process for their preparation, e.g. by admixing or coating.

The VLMWH compositions produced using the process of the invention may be used in concentrations or dosages comparable to those used for current LMWH, e.g. within 20% of the recommended levels for LMWH for the particular indication. Typical indications are described in the introductory portion of this text.

Viewed from a further aspect the invention provides a non-mammalian marine animal VLMWH composition having a VLMWH content, relative to total heparin content, of at least 10% wt, preferably at least 15% wt, more preferably at least 20% wt, especially at least 25% wt, more especially at least 30% wt (e.g. up to 100% wt, more typically up to 80% wt, for example up to 30% wt), optionally containing a physiologically acceptable carrier or excipient and/or a drug substance and optionally coated onto a substrate.

Viewed from a still further aspect the invention provides the use of a composition according to the invention or produced according to the process of the invention, in medicine, e.g. in compositions or equipment used in surgery, therapy, prophylaxis, or diagnosis on human or non-human animal subjects or for blood contact.

The invention will now be described further with reference to the following non-limiting Examples.

### EXAMPLE 1

### Production of Marine UFH

Equal amounts of tissue (salmon gills or waste) and buffer (5 mM NH₄CO₃/NH₃ in 0.1 M NaCl, pH 9.0) was homogenized in a tissue grinder (kitchen utility type, Braun). Typically, 300 g tissue in 300 ml buffer was used. The homogenate was incubated at 80° C. for 1 hour and centrifuged at 13000 rpm. The supernatant was applied onto a Dowex (2×8, anion exchanger), which was equilibrated in the buffer above and washed with the same buffer. Heparin was eluted using 4 M NaCl in the same buffer. This eluate was concentrated and desalted in a stirred cell (Amicon 8400) with a Nanomax-50 filter (MW cut-off=1000 Da). The concentrated and desalted eluate was freeze dried.

### EXAMPLE 2

### Production of Marine VLMWH

The heparin eluate from the Dowex anion exchange column, 100 ml in 4 M NaCl, of Example 1 (salmon waste) was filtered on a membrane with 100 Da MW cut-off (Omega 1K, Ultrasette membrane from Filtron/Pall) using a Millipore Masterflex pump with 1-2 ml/min. This system takes advantage of the principle of tangential flow.

The filtrate (i.e. the liquid which passed through the filter) was diluted 10 times in 5 mM NH₄CO₃/NH₃, pH 9.0, and desalted and concentrated in the stirred cell with a Nanomax-50 filter (1000 Da MW cut-off). The desalted concentrate was freeze dried. The freeze dried and desalted filtrate was dissolved in 1 ml of 0.025 M NH₄CO₃/NH₃, pH 9.0 and submitted to size exclusion chromatography on G-75 Sephadex (diameter 2.6 cm, 110 mL, and void volume 42 mL as determined with Blue Dextran), using 0.025 M NH₄CO₃/NH₃, pH 9.0 as the mobile phase.

By collecting the eluate after the first 47 mL of eluate has eluted from the column, and subsequently freeze drying the collected eluate, heparin of which at least 15% wt. has a molecular weight below 3000 Dalton is produced. This VLMWH rich heparin composition has an anti-factor Xa activity of 116 U/mg.

### EXAMPLE 3

### Preparation of Marine VLMWH

Waste extract was prepared according to Example 1 but applied to the Dowex anion exchanger in 5.6 times excess of the resin capacity. The product was then subjected to size exclusion chromatography as in Example 2. 28.6% wt of the treated product (relative to total heparin) was LMWH and 22.0% wt was VLMWH.

### EXAMPLE 4

### Preparation of Marine VLMWH

A Minim apparatus (Pall/Filtron USA) was used with a 3000 Da MW cut-off filter (Omega Centramate Suspended Screen, OS005C11P1) to filter waste extract prepared as in Example 1.

For filtration on the Minim apparatus, the flow was set to 80 ml/min, the flow was then restricted with a tube-stopper to 4 ml/min and the eluate (waste) in 4M NaCl/5 mM NH₄HCO₃/NH₃, pH 9.0 was submitted to tangential flow filtration on the 3000 Da MW cut-off filter.

The filtrate was concentrated and desalted in the stirred cell with the 1000 Da MW cut-off filter (Nanomax-50) as described above and freeze-dried. The freeze-dried filtrate was applied on the Sephadex G-75 for molecular weight filtration as described in Example 2.

The molecular weight filtration on Sephadex G-75 of the filtrate from the 3000 Da MW cut-off filtration showed that heparin eluted corresponded to a MW of from 3000 Da down.

### EXAMPLE 5

### Infusion Studies

Two freeze dried extracts produced as described above were investigated, one (Sample A) with Mol Weight <8000 and the other (Sample B) with Mol Weight >8000. These were each dissolved in 5 mL distilled water and filtered through a Millipore filters Millex GP filter unit 0.22 μm, to yield clear, light brown extracts were obtained. The antifactor Xa activity was determined with the Stachrom Heparin assay from Stago, Asnieres, France with the instrument StaCompact (see Teien et al., Thromb Res 10: 399-410 (1977)). Sample A contained 7.0 antifactor Xa/ml, Sample B contained 10.4 antifactor Xa/ml.

Infusion studies were performed on three healthy female rabbits with a weight of 4.3 kg, anesthesized with Hypnorm Vet®. Rabbit no 1 received intravenously 4 ml of Sample A, totalling 28 antifactor Xa units, corresponding to 6.5 Antifactor Xa Upper kg body weight. Rabbit no 2 received 3.8 ml of Sample B totalling 39.5 antifactor Xa units, corresponding to 9.2 antifactor Xa U/kg body weight. Rabbit no 3 received Fragmin® Pharmacia corresponding to 52 antifactor Xa Upper kg body weight. Blood (1.8 ml) was drawn in vacutainer tubes containing 0.2 ml 0.129 M Na-citrate before the infusion, and at the times 5, 15, 30, 60 and 90 minutes after the injections. The samples were mixed, centrifuged 2000 g, 15 min at room temperature, and the assays were performed within 3.5 hours.

Compared to human plasma, the ΔOD per min in rabbit plasma without exogenous glucosaminoglycans, was found to be somewhat lower, corresponding to a higher mean antifactor Xa activity of 0.13 U (range 0.11-0.16). All measurements in rabbit plasma were therefore subtracted 0.13 antiXa U. In Table 2 below, the plasma concentrations found with the three preparations are shown. The time courses of the plasma concentrations found indicate that the half-life of piscine GAGs is prolonged compared with the half life of Fragmin®.

TABLE 2
AntiFXa activity U/ml rabbit plasma

| Minutes after | | | |
|---|---|---|---|
| infusion | Rabbit 1 | Rabbit 2 | Rabbit 3 |
| 0 | 0.00 | 0.00 | 0.00 |
| 5 | 0.14 | 0.28 | 0.96 |
| 15 | 0.15 | 0.28 | 0.63 |
| 30 | 0.14 | 0.26 | 0.49 |
| 60 | 0.13 | 0.24 | 0.32 |
| 90 | 0.10 | 0.21 | 0.19 |

1. A process for the production of a very low molecular weight heparin (VLMWH) composition having a VLMWH content, relative to total heparin content, of at least 10% wt, said process comprising chromatographically or chemically or by filtration reducing the relative proportion of heparin having a molecular weight above 8000 Da in a heparin composition extracted from a non-mammalian, vascularised marine animal.

2. A process as claimed in claim 1 for the production of a VLMWH composition having a VLMWH content, relative to total heparin content, of at least 20% wt.

3. A process as claimed in claim 1 wherein the relative proportion of heparin having a molecular weight above 3000 Da is reduced.

4. A process as claimed in claim 1 performed on a heparin composition extracted from post muscle-removal fish waste.

5. A process as claimed in claim 4 performed on salmon waste.

6. A process as claimed in claim 1 performed chromatographically.

7. A non-mammalian marine animal very low molecular weight heparin (VLMWH) composition having a VLMWH content, relative to total heparin content, of at least 10% wt., optionally containing a physiologically acceptable carrier or excipient and/or a drug substance, and optionally coated onto a substrate.

8. A composition as claimed in claim 7 having a VLMWH content, relative to total heparin content, of at least 20% wt.

9. (canceled)

10. A human foodstuff comprising a composition according to claim 7.

11. A human foodstuff comprising a composition produced by the process of claim 1.
